(19) United States
(12) Patent Application Publication
(10) Pub. No.: US 2016/0199409 A1
(43) Pub. Date: Jul. 14, 2016

(54) Oral Delivery Product

(71) Applicant: Pfizer Inc., New York, NY (US)

(72) Inventors: Michael A. Goode, Powhatan, VA (US); Anne Rehne Volke, Skovlunde (DK)

(73) Assignee: Pfizer Inc., New York, NY (US)

(21) Appl. No.: 14/994,914

(22) Filed: Jan. 13, 2016

Related U.S. Application Data
(60) Provisional application No. 62/103,166, filed on Jan. 14, 2015.

(51) Int. Cl. A61K 33/32; A61K 31/355; A61K 31/519; A61K 31/122; A61K 31/4188; A61K 31/592; A61K 31/015; A61K 31/07; A61K 31/455; A61K 31/51; A61K 31/675; A61K 31/714; A61K 31/197; A61K 33/30; A61K 33/18; A61K 33/04; A61K 33/24; A61K 9/00; A61K 31/593; A61K 31/375

(52) U.S. Cl. CPC A61K 33/32; A61K 31/375; A61K 31/355; A61K 31/519; A61K 31/122; A61K 31/4188; A61K 31/592; A61K 31/015; A61K 31/07; A61K 31/455; A61K 31/51; A61K 31/675; A61K 31/714; A61K 31/197; A61K 33/30; A61K 33/18; A61K 33/04; A61K 33/24; A61K 9/0056; A61K 31/593; A23L 1/304; A23L 1/302; A23V 2002/00

(57) ABSTRACT

This invention provides a nutritional supplement composition which is a vitamin mint that can be taken anytime or anywhere without water or other liquid which is usually required for swallowing a tablet. The present invention is a nutritional supplement composition which is a convenient, flavorful and enjoyable vitamin mint which can be ingested without water.

This application claims the benefit under 35 USC 119(e) of U.S. Provisional Application No. 62/103,166 filed on Jan. 14, 2015, the contents of which is hereby incorporated by reference in its entirety.

### FIELD OF INVENTION

This invention relates to nutritional supplement compositions. More particularly this invention provides a nutritional supplement composition which is a vitamin mint that can be taken anytime or anywhere without water or other liquid which is usually required for swallowing a tablet. The present invention is a nutritional supplement composition which is a convenient, flavorful and enjoyable vitamin mint which can be ingested without water. The consumer can actively dissolve the vitamin mint in his or her mouth or the consumer can chew and swallow the mint; water is not required to consume the vitamin mint.

### BACKGROUND OF THE INVENTION

It has long been established that a number of chemical compounds typically referred to as vitamins and minerals provide significant value to maintaining an individual in a healthy state and/or treating specific medical conditions even when supplied in relatively small amounts. The human body cannot synthesize most of the vitamins and minerals that are essential to maintaining the health of the human body. Thus, vitamins and minerals must be obtained from an external source. The two most common external sources are foods and nutritional supplements. As most people do not eat foods that consistently provide the necessary daily requirements of vitamins and minerals, vitamin and mineral nutritional supplementation has become a recognized method of meeting accepted medical and health standards.

Micronutrients are elements or compounds which are present in foods in small or trace amounts and includes vitamins, minerals, or other elements and compounds found in foods for which many have not yet qualified for a recommended daily allowance (RDA). The macronutrients consist of carbohydrates, fats, and proteins that supply nutrients and calories and mostly are consumed via food and dietary intake. Some micronutrients such as calcium, sodium, potassium, chloride, and phosphorus are consumed in relatively large amounts, while many others such as iron, iodine, and zinc are consumed in small amounts. Vitamins, such as B12 and folic acid, and minerals such as selenium, are consumed in very small or trace amounts. In as much as the human body does not synthesize many compounds which are essential to the human body, these specific vitamins and minerals can be obtained from only two sources: food and supplements.

Vitamin and mineral preparations may be administered to treat specific medical conditions or as general nutritional supplements. As there are a number of vitamins and minerals needed and the daily amounts needed are relatively small, it is convenient to administer mixtures of vitamins and minerals in tablet or capsule form as a general supplement. Typical daily dosages of commercially available multivitamin and mineral supplements are one or two tablets or capsules per day. It is not unusual for such compositions to include two dozen or more nutrients in addition to the excipients needed to make the dosage form.

Nutritional deficiencies occurring in adults are many and vary according to geographic setting and socioeconomic status of the individual. The primary source of all nutrients is food; however, many adults do not meet the RDA of essential micronutrients through food consumption. Thus, vitamin and mineral supplementation has become a recognized method of meeting accepted medical and health standards. In an effort to combat these sub-optimal vitamin levels, there have been a variety of nutritional supplements made available to the public.

Consumers are interested in multivitamin and mineral nutritional supplements in dosage forms other than tablets because some tablets are large and can be difficult to swallow. In addition, water or another liquid is required to consume nutritional supplement tablets. Alternative dosage forms for nutritional supplements include chewable tablets; however, many consumers do not like the hardness of chewable tablets and the difficulty of chewing them. Furthermore, consumers do not like the chalky or gritty texture of some chewable tablets. Powdered drink mixes are another dosage form for nutritional supplements; however, the drink mixes require water.

Clearly, there is a need for a new nutritional supplement dosage form which provides a convenient form that can be ingested anytime and anywhere without the need for water or other liquid which is required for swallowing a tablet or preparing a powdered drink mix. Therefore, the inventors wanted to provide the nutritional supplement composition of the present invention which is a convenient, flavorful and enjoyable vitamin mint which can be ingested without water. The consumer can actively dissolve in his or her mouth or the consumer can chew and swallow the mint. Water is not required to ingest the vitamin mint nutritional supplement composition.

### SUMMARY OF THE INVENTION

In one aspect of the invention is provided a composition for oral delivery to a subject comprising vitamins or minerals, wherein the composition can dissolve in the subject's mouth or can be chewed and swallowed by the subject.

In another aspect of the invention is provided a composition wherein the vitamins are selected from Vitamin A, beta-carotene, Vitamin C, Vitamin D, Vitamin E, Vitamin K, thiamine, riboflavin, niacin, Vitamin B1, Vitamin B6, folic acid, Vitamin B12, Biotin and pantothenic acid.

In yet another aspect of the invention is provided a composition wherein the minerals are selected from calcium, iron, phosphorous, iodine, magnesium, zinc, selenium, copper, manganese, chromium, molybdenum, chloride, potassium, boron, nickel, silicon, tin and vanadium.

In some embodiments, the minerals are selected from zinc, manganese, iodine, selenium, molybdenum and chromium.

In some embodiments, the caloric content of the composition is about 5 calories.

In some embodiments, the hardness of the composition is 17 kP to 40 kP.

In some embodiments, the composition further comprises one or more flavorings.

In some embodiments, the flavorings are spray-dried flavorings or encapsulated flavorings.

In some embodiments, the flavoring is a mint flavoring, wintergreen flavoring, lemon flavoring or raspberry flavoring

In some embodiments, the average disintegration rate of the composition is approximately 5 to 10 minutes according to USP dissolution tests for dietary supplements.

In some embodiments, the average disintegration rate of the composition is approximately 350 to 362 seconds according to USP dissolution tests for dietary supplements.

The inventors believe that the combination of hardness of the vitamin mints and the use of sweetening agents which do not undergo crosslinking contribute to the ability of the composition of the invention to dissolve without water in the user's mouth.

### DETAILED DESCRIPTION OF THE INVENTION

The multivitamin and mineral dosage form described herein are intended for consumers who want a convenient, flavorful and enjoyable nutritional supplement mint which can be ingested without water. The consumer can allow the mint to dissolve in his or her mouth or the consumer can chew and swallow the mint. Water is not required to ingest the vitamin mint nutritional supplement composition. There are presently no commercially available nutritional supplements in the form of a mint which can be either allowed to dissolve in the consumer's mouth or which can be chewed and swallowed.

The nutritional supplements of the present invention are designed to replenish vitamins and minerals that the body loses on a daily basis and to provide the body with a full range of nutrients that it needs for optimal functionality. Those vitamins and minerals can include Vitamin A, beta-carotene, Vitamin D, Vitamin E, Vitamin K, thiamin, riboflavin, niacin, Vitamin B6, folic acid, Vitamin B12, biotin, pantothenic acid, calcium, iron, phosphorous, iodine, magnesium, zinc, selenium, copper, manganese, chromium, molybdenum, chloride, potassium, boron, nickel, silicon, tin and vanadium.

Folic acid or folate (the anionic form) also known as vitamin B9 is necessary for the production and maintenance of new cells. This is especially important during periods of rapid cell division and growth such as infancy and pregnancy. Folate is needed to synthesize DNA bases (most notably thymine, but also purine bases) needed for DNA replication. Thus folate deficiency hinders DNA synthesis and cell division, affecting most notably bone marrow and cancer, both of which participate in rapid cell division. Since folate deficiency limits cell division, erythropoisis, production of red blood cells (RBCs) is hindered and may lead to anemia.

Vitamin B1, also referred to as thiamine, is a water-soluble substance with thiazole and pyrimadine rings joined by a methylene bridge and has a biological half-life in the body of about 15 days. Thiamin is essential for neural function and carbohydrate metabolism and is dosed in the form of a pharmaceutically acceptable vitamin B1 compound. As used herein, “pharmaceutically acceptable” is a component suitable for use in humans without undue side effects, such as irritation, toxicity, and allergic response. Pharmaceutically acceptable vitamin B1 compounds may include, but are not limited to thiamin chloride hydrochloride.

Niacin is required for cell respiration, helps in the release of energy and metabolism of carbohydrates, fats, and proteins, proper circulation and healthy skin, functioning of the nervous system, and normal secretion of bile and stomach fluids. It is used in the synthesis of sex hormones, treating schizophrenia and other mental illnesses, and a memory-enhancer. Niacin given in pharmaceutical dosage improves the blood cholesterol profile, and has been used to clear the body of organic poisons, such as certain insecticides. A preferred form of niacin is niacinamide.

Vitamin B6 or pyridoxine is involved in the production of RNA and DNA and many other biological reactions in the human body. Pyridoxal phosphate, the metabolically active form of vitamin B6, is involved in many aspects of macronutrient metabolism, neurotransmitter synthesis, histamine synthesis, hemoglobin synthesis and function and gene expression. Pharmaceutically acceptable vitamin B6 compounds may include, but are not limited to pyridoxine, pydroxal and pyridoxamine, or salts thereof, including but not limited to pyridoxine HCl. The phosphate ester derivative pyridoxal phosphate generally serves as a coenzyme for many reactions and can help facilitate decarboxylation, transamination, racemization, elimination, replacement and beta-group interconversion reactions. An overdose of pyridoxine can cause a temporary deadening of certain nerves such as the proprioceptory nerves; causing a feeling of disembodiment common with the loss of propioception. This condition is reversible when supplementation is stopped.

Vitamin B12, or the cobalamins, is necessary for overall metabolism, nervous system function, metabolism of folic acid, homocysteine reduction and the production of red blood cells. There are at least three active forms of cobalamin: cyanocobalamin, hydroxocobalamin, and nitrocobalamin.

Biotin is necessary for the metabolism of carbohydrates, proteins, and fats and is needed for healthy skin and hair.

Pantothenic acid, also called vitamin B5, is a water-soluble vitamin required to sustain life. Pantothenic acid is needed to form coenzyme-A (CoA), and is critical in the metabolism and synthesis of carbohydrates, proteins, and fats. The derivative of pantothenic acid, pantothenol, is a more stable form of the vitamin and is often used as a source of the vitamin in multivitamin supplements. Another common supplemental form of the vitamin is calcium pantothenate. Calcium pantothenate is often used in dietary supplements because as a salt, it is more stable than pantothenic acid in the digestive tract allowing for better absorption. Calcium pantothenate may also reduce the duration of morning stiffness, degree of disability, and pain severity in rheumatoid arthritis patients. A preferred form of pantothenic acid is calcium pantothenate.

Vitamin E, a fat-soluble vitamin, is an antioxidant vitamin involved in the metabolism of all cells. It protects vitamin A and essential fatty acids from oxidation in the body cells and prevents breakdown of body tissues. Vitamin E is the generic term for a group of related substances that include alpha-tocopherol, beta-tocopherol, gamma-tocopherol, and delta-tocopherol. In addition, each of these four compounds has a “d” form, which is the natural form, and a “d1” form that is the synthetic form. Preferably, vitamin E is in the natural form.

Vitamin C, also known as ascorbic acid, is a water-soluble, antioxidant vitamin. It is important in forming collagen, a protein that gives structure to bones, cartilage, muscle, and blood vessels. Vitamin C also aids in the absorption of iron, and helps maintain capillaries, bones, and teeth. As a water-soluble antioxidant, vitamin C is in a unique position to scavenge aqueous peroxyl radicals before these destructive substances have a chance to damage lipids. It works along with vitamin E, a fat-soluble antioxidant, and the enzyme glutathione peroxidase to stop free radical chain reactions.

Vitamin C can enhance the body's resistance to an assortment of diseases, including infectious disorders and many types of cancer. It strengthens and protects the immune system by stimulating the activity of antibodies and immune system cells such as phagocytes and neutrophils. Vitamin C contributes to a variety of other biochemical functions. These include the biosynthesis of the amino acid carnitine and the catecholamines that regulate the nervous system. It also helps the body to absorb iron and to break down histamine. Although vitamin C is found in every cell, it is especially useful in key parts of the body. These include the blood, the skin, the nervous system, the teeth and bones and glands such as the thymus, adrenals and thyroid.

Vitamin D is a group of fat-soluble prohormones, the two major forms of which are vitamin D₂ (or ergocalciferol) and vitamin D₃ (or cholecalciferol). The term vitamin D as used herein also refers to metabolites and other analogues of these substances. Vitamin D₃ is produced in skin exposed to sunlight, specifically ultraviolet B radiation.

Vitamin D plays an important role in the maintenance of organ systems. It has been shown to regulate the calcium and phosphorus levels in the blood by promoting their absorption from food in the intestines, and by promoting re-absorption of calcium in the kidneys, which enables normal mineralization of bone. It is also needed for bone growth and bone remodeling. Vitamin D also has been suggested to affect the immune system by promoting phagocytosis, anti-tumor activity, and immunomodulatory functions.

Vitamin D deficiency can result from inadequate intake coupled with inadequate sunlight exposure, disorders that limit its absorption, conditions that impair conversion of vitamin D into active metabolites, such as liver or kidney disorders, or, rarely, by a number of hereditary disorders. Deficiency results in impaired bone mineralization, and leads to bone softening diseases, rickets in children and ostemalacia in adults, and possibly contributes to osteoporosis. However, sunlight exposure, to avoid deficiency, carries other risks, including skin cancer; which risk may be avoided with dietary absorption, either through diet or as a dietary supplement.

Zinc is an essential mineral that is naturally present in some foods, added to others, and available as a dietary supplement. Zinc is involved in numerous aspects of cellular metabolism. It is required for the catalytic activity of approximately 100 enzymes and it plays a role in immune function, protein synthesis, wound healing, DNA synthesis and cell division. A daily intake of zinc is required to maintain a steady state because the body has no specialized zinc storage system. Zinc deficiency is characterized by growth retardation, loss of appetite, and impaired immune function. In more severe cases, zinc deficiency causes hair loss, diarrhea, delayed sexual maturation, impotence, hypogonadism in males, and eye and skin lesions. Weight loss, delayed healing of wounds, taste abnormalities, and mental lethargy can also occur.

Severe zinc deficiency may also depress immune function, and even mild to moderate degrees of zinc deficiency can impair macrophage and neutrophil functions, natural killer cell activity, and complement activity. The body requires zinc to develop and activate T-lymphocytes. Individuals with low zinc levels have shown reduced lymphocyte proliferation response to mitogens and other adverse alterations in immunity that can be corrected by zinc supplementation. These alterations in immune function might explain why low zinc status has been associated with increased susceptibility to pneumonia and other infections. Several forms of zinc, including zinc gluconate, zinc sulfate, zinc oxide and zinc acetate may be used in the nutritional supplements described herein. The preferred form of zinc is zinc oxide.

Selenium is an essential trace element that functions as a component of enzymes involved in antioxidant protection and thyroid hormone metabolism. Selenium possesses antioxidant properties, and has been shown to reduce the risk of heart attack and heart disease. Characteristic signs of selenium deficiency have not been described in humans, but very low selenium status is a factor in the etiologies of a juvenile cardiomyopathy (Keshan Disease) and a chondrodystrophy (Kashin-Beck Disease) that occur in selenium-deficient regions of China.

Vitamin A, a bi-polar molecule formed with bi-polar covalent bonds between carbon and hydrogen, is linked to a family of similarly shaped molecules, the retinoids. Its important part is the retinyl group, which can be found in several forms. Vitamin A can be found as an ester, primarily retinyl palmitate (found in foods and converted to retinol in the small intestine). Vitamin A can also exist also as retinal or as retinoic acid. Precursors to the vitamin are present in foods of plant origin as some of the members of the carotenoid family of compounds. Common provitamin A carotenoids found in foods that come from plants are betacarotene, alpha carotene and betacryptoxanthin. Among these, beta-carotene is most efficiently made into retinol. Alpha carotene and betacryptoxanthin are also converted to vitamin A, but only half as efficiently as betacarotene.

Vitamin A plays a role in a variety of functions throughout the body, such as: vision, gene transcription, immune function, embryonic development and reproduction, bone metabolism, haematopoiesis, skin health, reducing risk of heart disease, and antioxidant activity. Vitamin A deficiency can occur as either a primary or secondary deficiency. A primary vitamin A deficiency occurs among children and adults who do not consume an adequate intake of yellow and green vegetables, fruits and liver. Secondary vitamin A deficiency is associated with chronic malabsorption of lipids, impaired bile production and release, low fat diets, and chronic exposure to oxidants, such as cigarette smoke. Vitamin A is a fat soluble vitamin and depends on micellar solubilization for dispersion into the small intestine, which results in poor utilization of vitamin A from low-fat diets. Zinc deficiency can also impair absorption, transport, and metabolism of vitamin A because it is essential for the synthesis of the vitamin A transport proteins and the oxidation of retinol to retinal.

Vitamin K denotes a group of lipophilic, hydrophobic vitamins that are need for postranslational modification of certain proteins. Chemically they are 2-methyl-1,4-napthoquinone derivatives. All members of the vitamin K group of vitamins share a methylated napthoquinone ring structure, and vary in the aliphatic side chain attached at the 3-position. Phylloquinone (also known as vitamin K₁) invariably contains in its side chain four isoprenoid residues, one of which is unsaturated.

It is generally accepted that the naphthoquinone is the functional group, so that the mechanism of action is similar for all K-vitamins. Substantial differences may be expected, however, with respect to intestinal absorption, transport, tissue distribution, and bio-availability. These differences are caused by the different lipophilicity of the various side chains, and by the different food matrices in which they occur. Vitamin K deficiency is very rare. It occurs when the body can't properly absorb the vitamin from the intestinal tract. Vitamin K deficiency can also occur after long-term treatment with antibiotics. Individuals with vitamin K deficiency are usually more likely to have bruising and bleeding. Vitamin K is an active blood clotting agent and assist in bone formation. Preferably, vitamin K is in the form of vitamin K1.

Additional micronutrients include but not limited to phosphorus, chloride, chromium, iodine, magnesium, manganese, molybdenum, and potassium.

Chromium assists in the regulation of glucose metabolism, is used in the synthesis of fatty acids and cholesterol, assists in transporting proteins, lowers LDL blood levels, and raises high density lipoproteins blood levels. Chromium may be dosed in a pharmaceutically acceptable chromium compound. Pharmaceutically acceptable chromium compounds may include, but are not limited to, chromium chloride, yeast-bound chromium, picolinate, niacin-bound chromium, and combinations thereof.

Iodine helps to metabolize fats and is necessary for proper thyroid function and helps to reduce fibrocystic breast conditions. Pharmaceutically acceptable forms of iodine, may include but are not limited to potassium iodide, sodium iodide and combinations thereof. A preferred form of iodine is potassium iodide.

Molybdenum may facilitate proper metabolism of fats, carbohydrate and iron and may protect against certain cancers. Pharmaceutically acceptable molybdenum compounds may include, but are not limited to, sodium molybdate, molybdenum amino acid chelates, and combinations thereof.

A low level of manganese in the body may be associated with diabetes. Accordingly manganese appears to have a role in the control of blood sugar levels. Manganese may also have a role in the metabolism of amino acids and certain vitamins. Pharmaceutically acceptable manganese compounds may include, but are not limited to, manganese chloride, manganese sulfate, and combinations thereof.

The term “mint” as used herein describes an oral dosage form that can be ingested anytime or anywhere without water or other liquid. A consumer can allow the mint to dissolve in his or her mouth or the consumer can chew and swallow the mint; water is not required to consume the vitamin mint.

Furthermore, these compositions can be made using conventional equipment and techniques known in the art. When preparing dosage forms incorporating the compositions of the invention, the nutritional components are normally blended with conventional excipients such as binders, including gelatin, pregelatinized starch, and the like; lubricants, such as hydrogenated vegetable oil, stearic acid, and the like; diluents, such as lactose, mannose, and sucrose; disintegrants, such as carboxymethyl cellulose and sodium starch glycolate; suspending agents, such as povidone, polyvinyl alcohol and the like; absorbents, such as silicon dioxide; and colorants, such as F.D. & C dyes and the like. In the present invention, binders may include sugar alcohols such as sorbitol, xylitol, mannitol, lactitol, maltitol, erythritol and isomalt. Sugar alcohols are used in the preferred embodiments due to their low water activity as well as their low caloric content. Because of their low caloric content, sugar alcohols are less likely to promote tooth decay than other binders. The flavorings used in the present invention may be spray-dried or encapsulated. The oral dosage forms may further contain sweetening agents such as acesulfame potassium, aspartame, sucralose, stevia and the like and/or flavoring agents such as essential oils, aldehydes, ketones, organic acids and the like.

Furthermore, the compositions may comprise additional micronutrients to supplement the daily dietary intake of those micronutrients. Those vitamins and minerals may include Vitamin A, beta-carotene, Vitamin D, Vitamin E, Vitamin K. thiamin, riboflavin, niacin, Vitamin B6, folic acid, Vitamin B12, biotin, pantothenic acid, calcium, iron, phosphorous, iodine, magnesium, zinc, selenium, copper, manganese, chromium, molybdenum, chloride, potassium, boron, nickel, silicon, tin and vanadium.

While the invention has been described with respect to certain specific embodiments, it will be appreciated that many modifications and changes may be made by those skilled in the art without departing from the invention. It is intended, therefore, by the appended claims to cover all such modifications and changes as may fall within the true spirit and scope of the invention.

### EXAMPLES

### Example 1

### Preparation of the Cool Mint Flavored Formulation

The Cool Mint formulation was prepared by combining the ingredients listed in the table below as well as excipients, flavorings, sweeteners and colorings. The mixture was then compressed and embossed to form the Cool Mint flavored nutritional supplement of the invention.

TABLE 1

| | Amount/ | | | |
|---|---|---|---|---|
| Nutrient | Mint | | Amount/2 Mints | |
| Vitamin C | 30 | mg | 60 | mg |
| Vitamin E | 16 | IU | 30 | IU |
| Folic Acid | 200 | mcg | 400 | mcg |
| Vitamin K | 32.5 | mcg | 65 | mcg |
| Biotin | 30 | mcg | 60 | mcg |
| Vitamin D | 500 | IU | 1000 | IU |
| BetaCarotene | 362.5 | IU | 725 | IU |
| Vitamin A | 887.5 | IU | 1775 | IU |
| Niacin | 1 | mg | 2 | mg |
| Vitamin B1 | 0.6 | mg | 1.2 | mg |
| Vitamin B6 | 1 | mg | 2 | mg |
| Vitamin B12 | 2.5 | mcg | 5 | mcg |
| Pantothenic acid | 2.5 | mg | 5 | mg |
| Zinc | 0.75 | mg | 1.5 | mg |
| Manganese | 1.15 | mg | 2.3 | mg |
| Iodine | 75 | mcg | 150 | mcg |
| Selenium | 10 | mcg | 20 | mcg |
| Molybdenum | 20 | mcg | 40 | mcg |
| Chromium | 17.5 | mcg | 35 | mcg |

### Example 2

### Preparation of the Wintergreen Flavored Formulation

The Wintergreen formulation was prepared by combining the ingredients listed in the table below as well as excipients, flavorings, sweeteners and colorings. The mixture was then compressed and embossed to form the Wintergreen flavored nutritional supplement of the invention.

TABLE 2

| | Amount/ | | | |
|---|---|---|---|---|
| Nutrient | Mint | | Amount/2 Mints | |
| Vitamin C | 30 | mg | 60 | mg |
| Vitamin E | 16 | IU | 30 | IU |
| Folic Acid | 200 | mcg | 400 | mcg |
| Vitamin K | 32.5 | mcg | 65 | mcg |
| Biotin | 30 | mcg | 60 | mcg |
| Vitamin D | 500 | IU | 1000 | IU |
| BetaCarotene | 362.5 | IU | 725 | IU |
| Vitamin A | 887.5 | IU | 1775 | IU |
| Niacin | 1 | mg | 2 | mg |
| Vitamin B1 | 0.6 | mg | 1.2 | mg |
| Vitamin B6 | 1 | mg | 2 | mg |
| Vitamin B12 | 2.5 | mcg | 5 | mcg |
| Pantothenic acid | 2.5 | mg | 5 | mg |
| Zinc | 0.75 | mg | 1.5 | mg |
| Manganese | 1.15 | mg | 2.3 | mg |
| Iodine | 75 | mcg | 150 | mcg |
| Selenium | 10 | mcg | 20 | mcg |
| Molybdenum | 20 | mcg | 40 | mcg |
| Chromium | 17.5 | mcg | 35 | mcg |

### Example 3

### Preparation of the Lemon Flavored Formulation

The Lemon formulation was prepared by combining the ingredients listed in the table below as well as excipients, flavorings, sweeteners and colorings. The mixture was then compressed and embossed to form the Lemon flavored nutritional supplement of the invention.

TABLE 3

| | Amount/ | | | |
|---|---|---|---|---|
| Nutrient | Mint | | Amount/2 Mints | |
| Vitamin C | 30 | mg | 60 | mg |
| Vitamin E | 16 | IU | 30 | IU |
| Folic Acid | 200 | mcg | 400 | mcg |
| Vitamin K | 32.5 | mcg | 65 | mcg |
| Biotin | 30 | mcg | 60 | mcg |
| Vitamin D | 500 | IU | 1000 | IU |
| BetaCarotene | 362.5 | IU | 725 | IU |
| Vitamin A | 887.5 | IU | 1775 | IU |
| Niacin | 1 | mg | 2 | mg |
| Vitamin B1 | 0.6 | mg | 1.2 | mg |
| Vitamin B6 | 1 | mg | 2 | mg |
| Vitamin B12 | 2.5 | mcg | 5 | mcg |
| Pantothenic acid | 2.5 | mg | 5 | mg |
| Zinc | 0.75 | mg | 1.5 | mg |
| Manganese | 1.15 | mg | 2.3 | mg |
| Iodine | 75 | mcg | 150 | mcg |
| Selenium | 10 | mcg | 20 | mcg |
| Molybdenum | 20 | mcg | 40 | mcg |
| Chromium | 17.5 | mcg | 35 | mcg |

### Example 4

### Preparation of the Raspberry Flavored Formulation

The Raspberry formulation was prepared by combining the ingredients listed in the table below as well as excipients, flavorings, sweeteners and colorings. The mixture was then compressed and embossed to form the Raspberry nutritional supplement of the invention.

TABLE 4

| | Amount/ | | | |
|---|---|---|---|---|
| Nutrient | Mint | | Amount/2 Mints | |
| Vitamin C | 30 | mg | 60 | mg |
| Vitamin E | 16 | IU | 30 | IU |
| Folic Acid | 200 | mcg | 400 | mcg |
| Vitamin K | 32.5 | mcg | 65 | mcg |
| Biotin | 30 | mcg | 60 | mcg |
| Vitamin D | 500 | IU | 1000 | IU |
| BetaCarotene | 362.5 | IU | 725 | IU |
| Vitamin A | 887.5 | IU | 1775 | IU |
| Niacin | 1 | mg | 2 | mg |
| Vitamin B1 | 0.6 | mg | 1.2 | mg |
| Vitamin B6 | 1 | mg | 2 | mg |
| Vitamin B12 | 2.5 | mcg | 5 | mcg |
| Pantothenic acid | 2.5 | mg | 5 | mg |
| Zinc | 0.75 | mg | 1.5 | mg |
| Manganese | 1.15 | mg | 2.3 | mg |
| Iodine | 75 | mcg | 150 | mcg |
| Selenium | 10 | mcg | 20 | mcg |
| Molybdenum | 20 | mcg | 40 | mcg |
| Chromium | 17.5 | mcg | 35 | mcg |

### Example 5

### Optimization of Vitamin Mint Hardness

The hardness of the vitamin mint of the invention was tested to determine the optimal hardness for the mint to maximize the consumer experience of allowing the mint to dissolve in his or her mouth or to chew and swallow the vitamin mint.

Stage 1: In the first stage, the overall hardness of marketed products in the United States was measured. The results of this study are given in Table 5

TABLE 5
Hardness of U.S. Marketed Products

| Product Name | Hardness Value (kP) |
|---|---|
| Certs Spearmint | 35.9 |
| BreathSaver | 43.1 |
| Peppermint | |
| LifeSaver | 22.3 |
| (Traditional) | |
| IceBreakers Mints | 44.8 |
| Altoids Mints | 23.3 |
| SweetTarts | 22.8 |

Based on the hardness measurements for the U.S. Marketed Products, a direct internal sensory consumer study was carried out to determine the optimal hardness for the vitamin mint of the invention.

Stage 2: In this stage, vitamin mints were prepared with a range of hardness from 13 kP to 30 kP and consumer reaction to the vitamin mint hardness was measured. The rating scale for the consumer experience was entitled the “Just About Right Hardness Rating Scale” to measure the full range of the consumer's opinion regarding the hardness of the vitamin mint, including (a) “Much Too Soft,” (b) “Just About Right,” and (c) “Much Too Hard.” Table 6 shows the results of the consumer test related to the Cool Mint flavored vitamin mint hardness.

TABLE 6

| | Tablet Hardness | | | | |
|---|---|---|---|---|---|
| | 1—(Much | | 3—(Just | | 5—(Much |
| Range | Too Soft) | 2 | About Right) | 4 | Too Hard) |
| 13-15 kP | 5 | 20 | 69 | 6 | 0 |
| 20-25 kP | 2 | 12 | 68 | 18 | 0 |
| 30 kP | 0 | 6 | 57 | 31 | 6 |

Based on the results of the consumer testing, the following Vitamin Mint Hardness Ranges were determined. The acceptable vitamin mint hardness range was 17 kilopond (kP) to 28 kP. The proposed vitamin hardness range was 17 kP to 40 kP. The inventors believe, without being bound to any theory, that they have achieved the optimal hardness of the vitamin mint which will improve the consumer experience. The results of this study bolster the enjoyment of the consumer in ingesting the vitamin mint of the invention either by letting the vitamin mint dissolve in his or her mouth or chewing and swallowing the vitamin mint.

### Example 6

### Consumer Testing

Consumer testing of numerous attributes of the Cool Mint flavored vitamin mint of the invention were performed. Table 7 lists the data from the consumer testing. The attributes that were tested included (1) aroma, (2) overall liking, (3) overall flavor liking, (4) flavor intensity liking, (5) sweetness, (6) hardness, (7) overall texture and (8) cooling effect. Liking scale ranged from 1 for “dislike extremely” to 9 for “like extremely.” The success criterion for the consumer tests was a rating of 6 or higher in overall liking. The ratings for the attributes tested given in Table 7 demonstrate that the success criterion was achieved for each attribute.

TABLE 7

| | Cool | | | | |
|---|---|---|---|---|---|
| | Mint | | Raspberry | Cool Mint | Lemon |
| | Vitamin | Wintergreen | Vitamin | Vitamin | Vitamin |
| | Mint | Vitamin Mint | Min | Mint | Mint |
| Attribute | (US) | (US) | (US) | (Italy) | (Italy) |
| Aroma | 7.2 | 7.0 | 6.8 | 7.2 | 6.2 |
| Overall | 6.8 | 6.9 | 5.7 | 6.6 | 6.7 |
| Liking | | | | | |
| Overall | 7.1 | 7.1 | 5.7 | 6.9 | 6.8 |
| Flavor | | | | | |
| Liking | | | | | |
| Flavor | 6.7 | 6.5 | 5.9 | 6.8 | 6.5 |
| Intensity | | | | | |
| Liking | | | | | |
| Sweetness | 6.6 | 6.4 | 6.0 | 6.7 | 6.3 |
| Hardness | 7.0 | 7.0 | 7.0 | 6.7 | 6.3 |
| Overall | 7.2 | 6.8 | 6.8 | 6.9 | 6.4 |
| Texture | | | | | |
| Cooling | 7.3 | 6.5 | 5.0 | 7.4 | 6.3 |
| Effect | | | | | |

Additional consumer testing was performed related the following attributes: (1) aroma, (2), grittiness, (3) chalkiness, (4) hardness, (5) coloring effect and (6) flavor intensity. The results are given in Table 8. The scale used in this test was a “Just About Right Scale” which is given as a percentage. The rating ranged from a 1 rating being “too little” to a 5 rating being “too much.” The values given in Table 8 show only the results for the 3 rating which is the “just about right” rating. The results showed the “just about right” rating was achieved the majority of the time for all the attributes tested.

TABLE 8

| | Cool | | | | |
|---|---|---|---|---|---|
| | Mint | Wintergreen | Raspberry | | Lemon |
| | Vitamin | Vitamin | Vitamin | Cool Mint | Vitamin |
| | Mint | Mint | Mint | Vitamin Mint | Mint |
| Attribute | (US) | (US) | (US) | (Italy) | (Italy) |
| Aroma | 70.8 | 68.8 | 61.5 | 65.5 | 57.1 |
| Grittiness | 76.9 | 67.2 | 83.3 | 79.3 | 80.0 |
| Chalkiness | 83.1 | 78.7 | 91.0 | 62.1 | 71.4 |
| Hardness | 75.4 | 68.8 | 87.2 | 72.4 | 60.0 |
| Cooling | 70.8 | 62.3 | 34.6 | 75.9 | 51.4 |
| Effect | | | | | |
| Flavor | 58.5 | 62.3 | 56.4 | 75.9 | 57.1 |
| Intensity | | | | | |

### Example 7

### Disintegration Studies

Disintegration studies were performed on the vitamin mint of the invention in comparison to other currently marketed multivitamin and mineral products. The disintegration studies were carried out using the procedure in USP Chapter 2040 entitled “DISINTEGRATION AND DISSOLUTION OF DIETARY SUPPLEMENTS.” Table 9 provides the disintegration data as an average value in seconds. The Centrum Adults Chewable (orange flavor) and the Centrum Kids Chewable (orange flavor) had an average disintegration time of approximately 1200 to 1300 seconds. The vitamin mint of the invention had an average disintegration time of approximately 350 to 362 seconds. The average disintegration times for the vitamin mint of the invention are much lower than the average disintegration times for the marketed multivitamin and mineral products. The disintegration data shows that the vitamin mint of the invention can be actively dissolved in the consumer's mouth. This data supports the property of the vitamin mint of the invention to be actively dissolved in the consumer's mouth.

TABLE 9

| | Average |
|---|---|
| | Disintegration |
| Product | Time |
| Name | (seconds) |
| Centrum | 1284 |
| Adult | |
| Chewable | |
| (Orange) | |
| Centrum Kids | 1308 |
| Chewable | |
| (Orange) | |
| Cool Mint | 362 |
| Vitamin mint | |
| Wintergreen | 350 |
| Vitamin mint | |

### Example 8

### Caloric Content

Table 10 lists the caloric content data for numerous marketed products in comparison to the vitamin mint of the invention. The caloric content was obtained from the product labels for the marketed products and was measured for the Cool Mint flavored vitamin mint of the invention.

TABLE 10

| Product Name | Calories per piece |
|---|---|
| Brach Star Brite | 20 |
| Peppermint Mint | |
| Bob's Sweet Stripe | 20 |
| Soft Mint | |
| Curiously Strong | 9 |
| Altoids Spearment | |
| Lifesaver Wint O Green | 6.7 |
| Certs CoolMint Drops | 6 |
| Certs Wintergreen | 5 |
| Certs Peppermint | 5 |
| With Retsyn Crystals | |
| Dolce | 5 |
| Centrum Adult | 5 |
| Chewable (Orange) | |
| Centrum Silver Chewable | 5 |
| BreathSaver Wintergreen | 5 |
| BreathSaver 3 | 5 |
| Hour Spearmint | |

The caloric content data given in Table 10 demonstrate that the vitamin mints of the invention have a lower caloric content than some marketed products such as Brach Star Brite Peppermint Mint, Bob's Sweet Stripe Soft Min, Lifesave Wint O Green and Certs CoolMint Drops. The caloric content of the vitamin mints of the invention is comparable to the Centrum Adult Chewable (Orange) and Centrum Silver Chewable, among others.

### Example 9

### Vitamin Mint Hardness Study

Additional vitamin mint hardness studies were performed on the Cool Mint Flavored Formulation of Example 1 to evaluate the hardness of the mint at various temperatures, relative humidities and time points and in the presence or absence of desiccant. Package codes A, B and E indicate that the studies were performed without desiccants in the packaging. Package codes C and D indicate that the studies were performed with 1 gram of desiccant or 2 grams of desiccant in the packaging, respectively. Tables 11 and 12 list the hardness measurements for the vitamin mints under various conditions.

Table 11 lists the hardness measurements for the study which was carried out at 25° C. and 60% relative humidity at the 0, 6 and 12 month timepoints in both the absence and presence of desiccant. Table 12 lists the hardness measurements for the study which was carried out at 40° C. and 70% relative humidity at multiple timepoints in both the absence and presence of desiccant. The results show that the hardness of the vitamin mint did not change significantly over time under various storage and packaging conditions. This data indicates that the case hardness of the vitamin mint of the invention does not change significantly over time.

TABLE 11

| | Package | Timepoint | Hardness |
|---|---|---|---|
| Condition | Code | (Months) | (kP) |
| 25 C./60% RH | A | 0 | 20.0 |
| 25 C./60% RH | A | 6 | 23.9 |
| 25 C./60% RH | A | 12 | 23.6 |
| 25 C./60% RH | B | 0 | 20.0 |
| 25 C./60% RH | B | 6 | 24.1 |
| 25 C./60% RH | B | 12 | 23.6 |
| 25 C./60% RH | C | 0 | 20.0 |
| 25 C./60% RH | C | 6 | 21.9 |
| 25 C./60% RH | C | 12 | 23.9 |
| 25 C./60% RH | D | 0 | 20.0 |
| 25 C./60% RH | D | 6 | 24.1 |
| 25 C./60% RH | D | 12 | 24.1 |
| 25 C./60% RH | E | 0 | 20.0 |
| 25 C./60% RH | E | 6 | 19.8 |
| 25 C./60% RH | E | 12 | 23.4 |

TABLE 12

| | Package | Timepoint | Hardness |
|---|---|---|---|
| Condition | Code | (Months) | (kP) |
| 40 C./75% RH | A | 0 | 27.8 |
| 40 C./75% RH | A | 1 | 28.0 |
| 40 C./75% RH | A | 2 | 26.8 |
| 40 C./75% RH | A | 3 | 32.4 |
| 40 C./75% RH | A | 0 | 20.0 |
| 40 C./75% RH | A | 1.5 | 24.6 |
| 40 C./75% RH | A | 3 | 29.5 |
| 40 C./75% RH | A | 6 | 37.6 |
| 40 C./75% RH | B | 0 | 20.0 |
| 40 C./75% RH | B | 1.5 | 24.4 |
| 40 C./75% RH | B | 3 | 27.8 |
| 40 C./75% RH | B | 6 | 27.3 |
| 40 C./75% RH | C | 0 | 20.0 |
| 40 C./75% RH | C | 3 | 25.8 |
| 40 C./75% RH | C | 6 | 27.2 |
| 40 C./75% RH | D | 0 | 20.0 |
| 40 C./75% RH | D | 1.5 | 20.1 |
| 40 C./75% RH | D | 3 | 24.3 |
| 40 C./75% RH | D | 6 | 26.7 |
| 40 C./75% RH | E | 0 | 20.0 |
| 40 C./75% RH | E | 1.5 | 23.0 |
| 40 C./75% RH | E | 3 | 23.5 |
| 40 C./75% RH | E | 6 | 26.4 |

1. A composition for oral delivery to a subject comprising vitamins or minerals, wherein the composition can dissolve in the subject's mouth or can be chewed and swallowed by the subject.

2. The composition of claim 1 wherein the vitamins are selected from Vitamin C, Vitamin E, folic acid, Vitamin K, biotin, Vitamin D, betacarotene, Vitamin A, niacin, Vitamin B1, Vitamin B6, Vitamin B12 and pantothenic acid.

3. The composition of claim 1 wherein the minerals are selected from calcium, iron, phosphorous, iodine, magnesium, zinc, selenium, copper, manganese, chromium, molybdenum, chloride, potassium, boron, nickel, silicon, tin and vanadium.

4. The composition of claim 3 wherein the minerals are selected from zinc, manganese, iodine, selenium, molybdenum and chromium.

5. The composition of claim 1 further comprising binders selected from sorbitol, xylitol, mannitol, lactitol, maltitol, erythritol and isomalt.

6. The composition of claim 1 further comprising sweetening agents selected from acesulfame potassium, aspartame, sucralose and stevia.

7. The composition of claim 1 wherein the caloric content of the composition is about calories.

8. The composition of claim 1 wherein the hardness of the composition is 17 kp to 40 kP.

9. The composition of claim 1 further comprising one or more flavorings.

10. The composition of claim 9 wherein the flavorings are spray-dried flavorings or encapsulated flavorings.

11. The composition of claim 9 wherein the flavoring is a mint flavoring, wintergreen flavoring, lemon flavoring or raspberry flavoring.

12. The composition of claim 1 wherein the average disintegration rate of the composition is approximately 5 to 10 minutes.

13. The composition of claim 1 wherein the average disintegration rate of the composition is approximately 350 to 362 seconds.
